(19) United States
(12) Patent Application Publication
(10) Pub. No.: US 2017/0028170 A1
(43) Pub. Date: Feb. 2, 2017

(54) GUIDE CATHETER EXTENSION DEVICE AND METHODS OF USE FOR CARDIOLOGY PROCEDURES

(71) Applicant: Andrew Ho, M.D., Inc., Corona, CA (US)

(72) Inventor: ANDREW Ho, Corona, CA (US)

(21) Appl. No.: 15/222,455

(22) Filed: Jul. 28, 2016

Related U.S. Application Data
(60) Provisional application No. 62/282,157, filed on Jul. 28, 2015.

(51) Int. Cl. A61M 25/00; A61M 25/01; A61F 2/95; A61M 25/10; A61M 25/09

(52) U.S. Cl. CPC A61M 25/0068; A61M 25/104; A61M 25/09041; A61F 2/95; A61M 25/0113; A61M 25/0045; A61M 2025/0175; A61M 2025/0188; A61M 2025/0046

(57) ABSTRACT

A guide catheter extension device for use with a standard guide catheter. The guide catheter extension device is made up of a flexible, elongate extension catheter having a tapered tip portion at a distal end, an opening at a proximal end, and a body portion extending between the two. The extension catheter has a longitudinal slit extending from the distal tip portion toward the proximal opening. Methods of using the guide catheter extension device to aid in performing interventional cardiology procedures.

### CROSS-REFERENCE TO RELATED APPLICATIONS

This application claims the benefit of U.S. Provisional Application No. 62/282,157, titled GUIDE LINE WITH A NOVEL SPIRAL DESIGN, filed on Jul. 28, 2015, the teachings of which are expressly incorporated by reference.

### STATEMENT RE: FEDERALLY SPONSORED RESEARCH/DEVELOPMENT

Not Applicable

### BACKGROUND

The present disclosure relates generally to devices and methods for treating heart disease and more particularly to a guiding catheter extension device for aiding in the delivery of interventional cardiology devices to a treatment site within a patient.

In general, interventional cardiology procedures, including angioplasty, require inserting interventional cardiology devices through catheters into coronary arteries that branch off from the aorta. “Interventional cardiology devices” may include, but is not limited to, balloon catheters, stent catheters, and guide wires. Atherosclerosis is a disease affecting the coronary arteries, by narrowing or occluding the arteries due to the growth of atherosclerotic plaque on the inside wall of the artery. When the artery is partially narrowed, it is referred to as a stenosis. During cardiac procedures, it is often necessary to place an interventional cardiology device within, or through, the treatment site, i.e., the occlusion or stenosis.

For example, during balloon dilation angioplasty, a guide catheter is inserted into an artery of the patient, for example the femoral artery or an artery within the patient's arm, and guided through the artery to the aorta and into the ostium of the coronary artery to be treated. A guide wire is then inserted through a lumen of the guide catheter, and advanced through the guide catheter such that the guide wire extends out of the distal end of the guide catheter into the coronary artery to be treated and reaches, or passes though, the treatment site. A dilation balloon catheter is then threaded over the guide wire and run through the lumen of the guide catheter to reach the treatment site. Once disposed within the treatment site, the balloon is inflated to compress the plaque against the artery wall and to stretch the artery open, thereby resulting in an acceptable amount of blood flow through the artery to the heart.

In a variation of this procedure, the procedure further includes a stent wrapping around the balloon, such that when the balloon is inflated, the stent expands to the size of the artery and helps to physically hold open the artery once the balloon is deflated and removed from the patient. In this variation, the stent remains in place within the patient's artery after the conclusion of the procedure.

As the coronary arteries are already narrow, even before the presence of a stenosis or occlusion, it can be difficult to properly guide an interventional cardiology device to the proper location. This is particularly true if the device has to traverse through heavily calcified or tortuous coronary vessels to get to the site of stenosis.

Alternatively, the use of an overly rigid device catheter may potentially damage the blood vessels as it is being guided through them due to its rigidity.

In order to overcome these problems, there have been introduced various guide catheter extension devices that are disposed within the lumen of the external guide catheter, and that are configured to accept the interventional cardiology device within a lumen of the guide catheter extension device. These guide catheter extension devices typically have a higher rigidity than the interventional cardiology device, thereby giving support to the device, yet has a smaller cross-sectional diameter than that of the external guide catheter. This combination of increased rigidity and decreased diameter will often allow for the proper placement of the interventional cardiology device at the treatment site. Examples of prior guide catheter extension devices are described in U.S. Pat. No. 5,527,292 issued to Adams et al. and U.S. Pat. Nos. 8,048,032, 8,142,413, and 8,292,850 issued to Root et al. In particular, Adams discloses a guide catheter extension device having sufficient rigidity to be helpful in introducing an interventional cardiology device to a treatment site; however, the extension device described in Adams is configured in such a fashion that it may be difficult to get to, or pass through, narrow sections of a blood vessel as its diameter is constant. Furthermore, while Adams described a rounded distal tip portion, since it maintains a constant diameter, this tip may damage, or occlude, blood vessels it is being routed through.

In order to overcome this deficiency, Root describes extension devices that include a tapered distal tip portion to aid in atraumatic placement within the blood vessel. However, since the tapered tip portion is solid, having a lumen configured large enough to only contain a guide wire, Root requires that its tapered tip catheter be placed within yet another extension catheter (i.e., two extension devices, one sitting inside of the other, are utilized). Once the two extension catheters have been placed at the desired treatment site, the inner tapered catheter is removed, and the interventional cardiology device can be introduced into the lumen of the outer extension catheter retained within the patient's blood vessel. As can be seen, this requires numerous steps, and numerous parts, that render the use of the Root device problematic.

Furthermore, if the balloon or stent is introduced into the patient prior to the use of the extension device, and it is discovered that the balloon or stent catheter, on its own, will not be able to reach the treatment site, it must first be removed in order to insert the extension device into the patient. This can lead to an unwanted delay in the surgical procedure, and the requirement to once again route the device through the patient's tortuous blood vessels. While Adams describes an embodiment having a split down the length of the extension device, thereby eliminated the need to remove the balloon catheter from the patient in the case where the balloon catheter itself is not rigid enough to reach the surgical site, it still has the problem of being overly rigid with a large cross-sectional diameter that may injure the patient's blood vessels, or that may simply be too wide to reach the treatment site.

As such, there is a need for a guide catheter extension device that is sufficiently rigid to aid in the placement of interventional cardiology devices at the treatment site, that is capable of being used without first removing the interventional cardiology device from the patient if necessary, and that will be sufficiently narrow during its use to reach the treatment site, while still allowing the interventional cardiology device to be passed through it to reach the treatment site.

### BRIEF SUMMARY

In accordance with one embodiment of the present disclosure, there is contemplated a guide catheter extension device for use with a standard guide catheter. The guide catheter extension device is made up of a flexible elongate extension catheter having a tapered tip portion at a distal end, an opening at a proximal end, a body portion extending between the distal tip portion and the proximal opening, and a longitudinal slit extending from the distal tip portion toward the proximal opening. The extension catheter defines a lumen extending from the tip portion to the proximal opening. The guide catheter extension device further includes a push rod attached to the extension catheter at the proximal opening.

The extension catheter may be formed from a resilient material, such as a metal, plastic, or a composite structure. In particular, if the extension catheter is formed from a plastic, it may be formed from a polyolefin, polyethylene, or a polyurethane.

Due to the extension catheter being formed from a resilient material and having a longitudinal slit along its length, the extension catheter has a initial, smaller, resting diameter. The extension catheter may be deformed outward when subjected to internal pressure, such as by introducing an interventional cardiology device into the lumen of the extension catheter. The internal pressure within the extension catheter lumen forces the longitudinal slit open, such that the extension catheter now has a subsequent, larger, diameter. Once the internal pressure is removed, the extension catheter will return to its initial resting diameter.

In certain embodiments, the longitudinal slit extends the entire length of the extension catheter. In other embodiments, the longitudinal slit is only present at the tip portion, or otherwise extends less than the entire length of the extension catheter.

In certain embodiments the distal tip tapers from its narrowest diameter at its most distal point, and widens as it approaches the extension catheter body. The extension catheter body may extend to the proximal opening at approximately the same diameter along the length of the body. In other embodiments, the extension catheter tapers along its entire length from the distal tip to the proximal opening.

The push rod may be configured to be more rigid than the extension catheter. In particular, the push rod may be formed from a material such as a hypotube, stainless steel, or Nitinol tubing.

The guide catheter extension device is configured to be longer than the guide catheter. The extension catheter lumen and/or the outside of the extension catheter may be coated with a slippery substance. For example, the extension catheter may be coated with silicone or PTFE.

Other embodiments of the present disclosure contemplate methods for performing interventional cardiology procedures at a surgical site in a patient in need thereof. In one such method, a standard guide catheter is inserted into a coronary artery ostium of the patient. A guide wire is then inserted into a lumen of the guide catheter and the guide wire is advanced past a distal end of the guide catheter, into the coronary artery, and ultimately to the surgical site. A guide catheter extension device is then inserted into the guide catheter lumen, such that the guide wire is disposed within a lumen of the guide catheter extension device. The guide catheter extension device is made up of a flexible elongate extension catheter having a tapered tip portion at a distal end, an opening at a proximal end, and a body portion extending between the distal tip portion and the proximal opening. The extension catheter further defines a lumen extending from the tip portion to the proximal opening, and has a longitudinal slit extending from the distal tip portion toward the proximal opening. The guide catheter extension device further includes a push rod attached to the extension catheter at the proximal opening. The guide catheter extension device is then advanced past the distal end of the guide catheter, into the coronary artery, and to the surgical site. An interventional cardiology device to be used in the surgery is then inserted into the guide catheter extension device lumen and advanced past the tapered tip portion and to the surgical site, wherein the interventional cardiology may be utilized as is known in the art.

In particular, the interventional cardiology device may be a balloon catheter or a stent.

Yet another embodiment envisions another method for performing an interventional cardiology procedure at a surgical site in a patient in need thereof. In this embodiment, the standard guide catheter is similarly inserted into a coronary artery ostium of the patient, followed by the insertion of the guide wire into the guide catheter's lumen. The guide wire is advanced past the distal end of the guide catheter, into the coronary artery, and to the surgical site. However, in this embodiment, the interventional cardiology device is now inserted directly into the guide catheter lumen. The interventional cardiology device is now advanced past the guide catheter distal end, into the coronary artery, and toward the surgical site. If the interventional cardiology device happens to be blocked from reaching the surgical site, the surgeon can then at that point insert the guide catheter extension device into the guide catheter lumen, such that the interventional cardiology device is disposed within the guide catheter extension device's lumen.

The guide catheter extension device is made up of a flexible elongate extension catheter having a tapered tip portion at a distal end, an opening at a proximal end, and a body portion extending between the distal tip portion and the proximal opening. The extension catheter further defines a lumen extending from the tip portion to the proximal opening, and includes a longitudinal slit extending from the distal tip portion toward the proximal opening and a push rod attached to the extension catheter at the proximal opening. The guide catheter extension device is then advanced past a distal end of the interventional cardiology device, or at least abutting a proximal end of the interventional cardiology device. The interventional cardiology device can then be advanced, with the aid of the guide catheter extension device, to the surgical site.

As in the other methods, the interventional cardiology device may be a balloon or a stent catheter.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1A is a schematic view of a guide catheter extension device of the present disclosure;

FIG. 1B is a schematic view of another embodiment of a guide catheter extension device of the present disclosure;

FIG. 1C is a schematic view of yet another embodiment of a guide catheter extension device of the present disclosure;

FIG. 1D is a sectional view of the guide catheter extension device of the present disclosure being utilized with a guide wire, guide catheter, and balloon catheter;

FIG. 2A is a detailed view of the distal tip of the guide catheter extension device depicted in FIG. 1A;

FIG. 2B is a detailed view of the distal tip of the guide catheter extension device depicted in FIG. 1B;

FIG. 2C is a detailed view of the distal tip of the guide catheter extension device of the present disclosure being utilized with a guide wire and a balloon catheter;

FIG. 2D is a cross-sectional view of the guide catheter extension device of the present disclosure;

FIG. 2E is a cross-sectional view of the guide catheter extension device of the present disclosure as depicted in FIG. 1D;

FIG. 3 is a schematic view of a guide catheter inserted in an aortic arch and reaching the ostium of a coronary artery;

FIG. 4 is a schematic view of the guide catheter depicted in FIG. 3 with a guide wire inserted through the guide catheter and into the coronary artery past a lesion to be treated;

FIG. 5 is a schematic view of the guide catheter and guide wire depicted in FIG. 4 with an extension catheter of the present disclosure extended through the guide catheter to the lesion to be treated in the coronary artery;

FIG. 6A is a schematic view of a balloon catheter and balloon being inserted through the extension catheter toward the lesion;

FIG. 6B is a detailed view of the balloon and extension catheter depicted in FIG. 6A;

FIG. 7A is a schematic view of the balloon depicted in FIG. 6A being successfully located at the lesion;

FIG. 7B is a detailed view of the balloon depicted in FIG. 7A;

FIG. 8A is a schematic view of a balloon catheter extended through the guide catheter depicted in FIG. 4 with no extension catheter being utilized;

FIG. 8B is a detailed view of the balloon catheter depicted in FIG. 8A wherein the balloon is not properly located within the lesion for treatment;

FIG. 9A is a schematic view of the balloon catheter depicted in FIG. 8A, wherein the extension catheter of the present disclosure has been inserted through the guide catheter to encompass the balloon;

FIG. 9B is a detailed view of the balloon depicted in FIG. 9A contained within the extension catheter of the present disclosure;

FIG. 10A is a schematic view depicting the balloon being successfully located at the lesion; and

FIG. 10B is a detailed view of the balloon depicted in FIG. 10A.

### DETAILED DESCRIPTION

The detailed description set forth below is intended as a description of the presently preferred embodiment of the invention, and is not intended to represent the only form in which the present invention may be constructed or utilized. The description sets forth the functions and sequences of steps for constructing and operating the invention. It is to be understood, however, that the same or equivalent functions and sequences may be accomplished by different embodiments and that they are also intended to be encompassed within the scope of the invention.

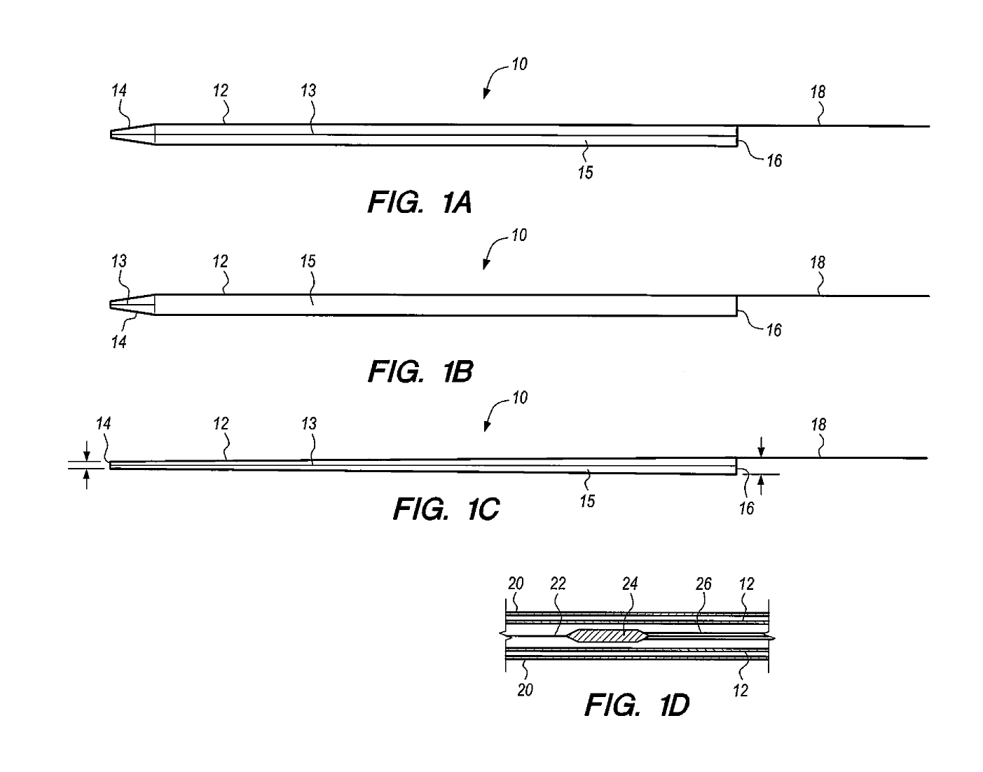

As shown in FIGS. 1A-D and 2A-E, the guide catheter extension device 10 of the present disclosure generally includes a flexible elongate extension catheter 12 having a longitudinal slit 13 extending from a distal tip 14 at one end of the extension catheter 12. The extension catheter 12 defines a lumen 28 extending though the distal tip 14, along a body portion 15 of the extension catheter 12, and toward a proximal opening 16 at the other end of the extension catheter 12. The guide catheter extension device 10 further includes a push rod 18 attached to the extension catheter 12 at, or near, the proximal opening 16.

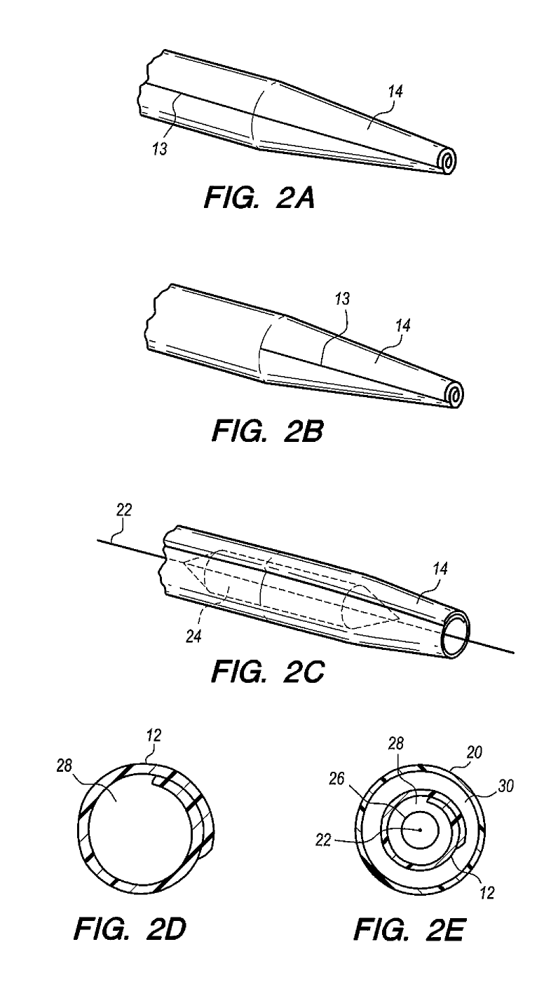

The extension catheter 12 is preferably formed from a soft, flexible springlike material, such as metal, plastic, or composite structures known to the art. Suitable examples of plastics capable of being used to form the extension catheter 12 include, but are not limited to, polyolefin, polyethylene, and polyurethane. The extension catheter 12 is formed such that at least a portion of the lumen 28 has a minimal inner diameter when at rest (as shown in FIGS. 2A and 2B), but is capable of deforming outward when under internal pressure due to the longitudinal slit 13 allowing the extension catheter 12 to open (as shown in FIG. 2C), and then returning to its resting state when no longer under internal pressure. For example, as shown in FIGS. 1D, 2C, and 2E, when an interventional cardiology device, such as a balloon 24, having an outer diameter larger than the extension catheter lumen 28 when at rest, is inserted within the extension catheter 12, the pressure applied by the balloon 24 causes the extension catheter lumen 28 to expand outwardly to encompass the balloon 24 due to the longitudinal slit 13. When the balloon 24 is no longer present within the lumen 28, the lumen 28 returns to its resting, minimal diameter. In other words, the longitudinal slit 13 uncurls when faced with internal pressure within the lumen 28, and due to the resilient nature of the extension catheter 12, the longitudinal slit 13 resumes its initial, resting position.

The longitudinal slit 13 may extend the entire length of the extension catheter 12, as shown in FIGS. 1A and 1C, or may only extend a portion of the extension catheter's length from the distal tip 14, as shown in FIG. 1B. One benefit of the longitudinal slit 13 extending the entire length of the extension catheter 12 is that if an interventional cardiology device 24 is already disposed within a patient being treated, and the device 24 is either obstructed before reaching the intended surgical site or needs to be swapped for another device, the extension catheter 12 can be placed over the device 24 by opening the longitudinal slit 13 and snapping the extension catheter 12 onto the device 24.

The distal tip 14 has a tapered shape such that it is narrowest at its most distal point, and widens as it approaches the extension catheter body 15. As shown in FIGS. 1A and 1B, only the distal tip 14 tapers, and the body 15 extends to the proximal opening 16 at approximately the same diameter along the length of the body 15. However, other configurations are envisioned, such as that depicted in FIG. 1C wherein the extension catheter 12 tapers along its entire length from the tip 14 having a smaller diameter 14A, along the body 15 all the way to the proximal opening 16 having a larger diameter 16A.

The push rod 18 is relatively rigid to allow the surgeon utilizing the device to maneuver the guide catheter extension device to the desired surgical site. The push rod 18 may be formed from a hypotube, or stainless steel, or Nitinol tubing, and can be attached to the extension catheter 12 by welding, bonding, or other methods known within the art.

The guide catheter extension device 10 is formed to an overall length appropriate for its use, but can be approximately 125 cm. Other lengths can be utilized as is appropriate. The guide catheter extension device 10 is, importantly, longer than the guide catheter 20.

As seen in FIGS. 1D and 2E, the extension catheter 12 is configured to fit within a lumen 30 of the guide catheter 20, such that the outer diameter of the extension catheter 12 is smaller than the inner diameter of the guide catheter lumen 30. Furthermore, the extension catheter 12 is configured to be placed around, and slidable along, both a guide wire 22, and an interventional cardiology device 24, such as a balloon catheter 26. The extension catheter lumen 28 and/or the outside of the extension catheter 12 may be coated with a slippery substance, such as silicone or PTFE, in order to aid in the movement within the guide catheter 20 or of the interventional cardiology device 24 within the extension catheter 12.

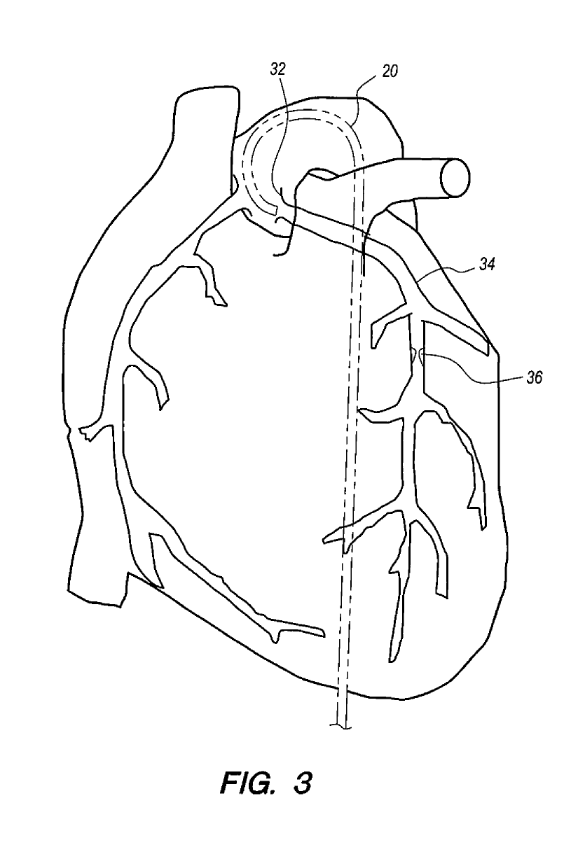

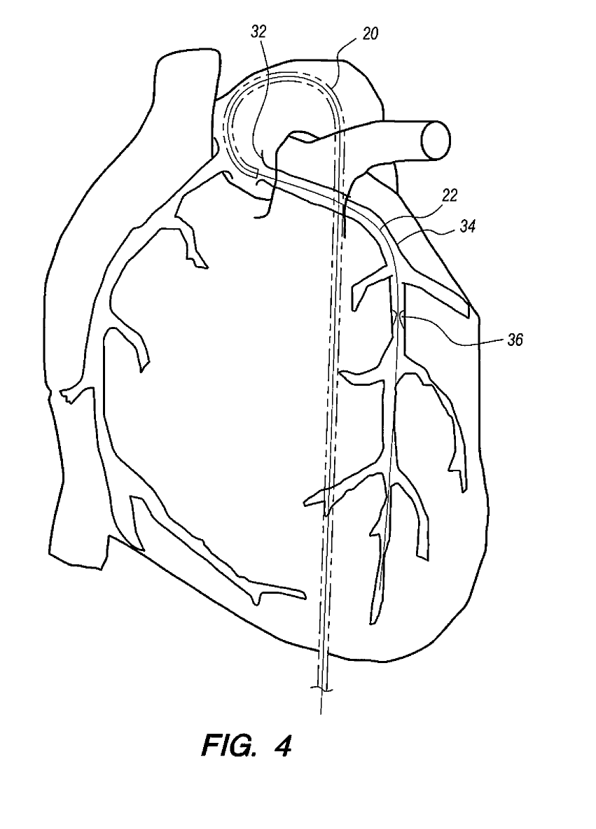

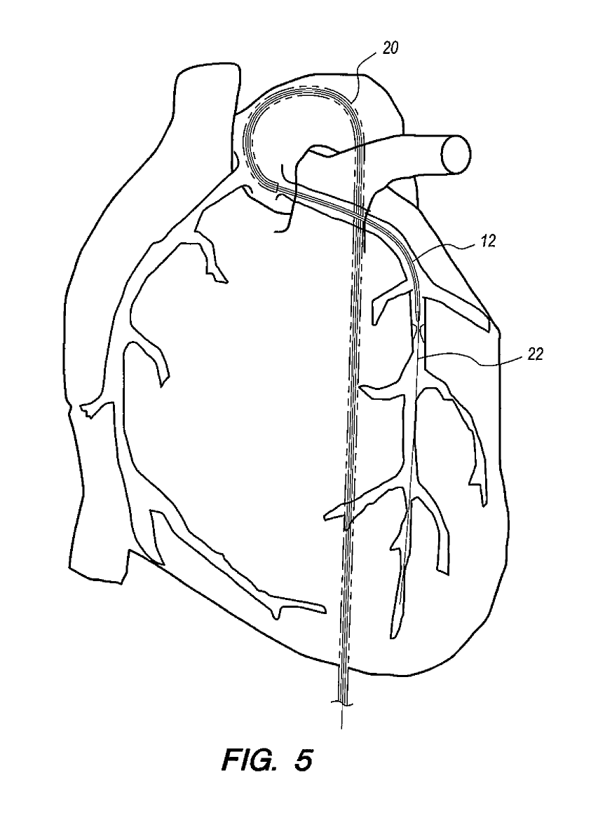

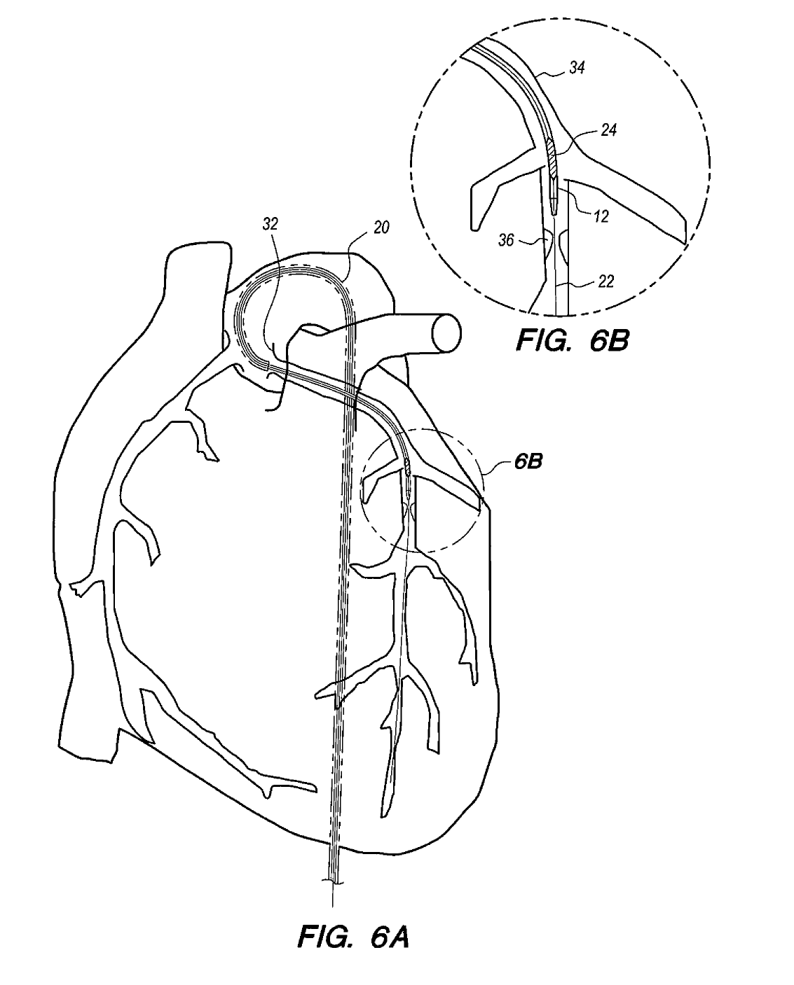

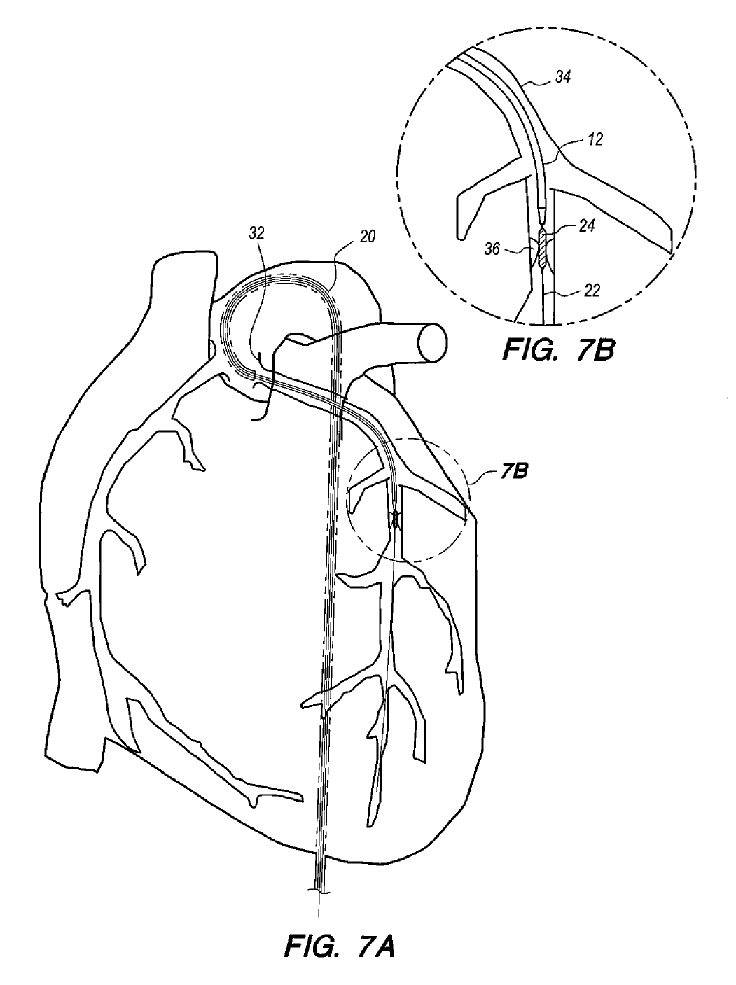

The guide catheter extension device 10 of the present disclosure may be utilized in two possible scenarios. In the first scenario, depicted in FIGS. 3-7, the guide catheter 20 is extended through the patient's aorta to the ostium 32 of a coronary artery 34 (shown in FIG. 3). Next, the guide wire 22 is inserted through the guide catheter lumen 30 and out of the guide catheter 20 into the coronary artery 34 to a surgical site 36, such as a lesion or stenosis (shown in FIG. 4). Then, as shown in FIG. 5, the extension catheter 12 is inserted into the guide catheter lumen 30, such that the guide wire 22 is disposed within the extension catheter lumen 28. The extension catheter 12 is then advanced through the guide catheter 20 until the extension catheter 12 reaches the surgical site 36. The extension catheter 12 may be maneuvered by the surgeon advancing the push rod 18. The minimal leading diameter of the extension catheter 12, due to its tapered distal tip 14, and curling inward from the longitudinal slit 13, allows the extension catheter 12 to be easily maneuvered through the coronary artery, while minimizing the possibility of injuring the artery. Once the extension catheter 12 is positioned properly, the interventional cardiology device 24, such as a balloon catheter 26, is advanced through the extension catheter lumen 28 (as shown in FIG. 6) until being positioned properly within the surgical site 36 (as shown in FIG. 7), at which point the surgeon can proceed to treat the patient.

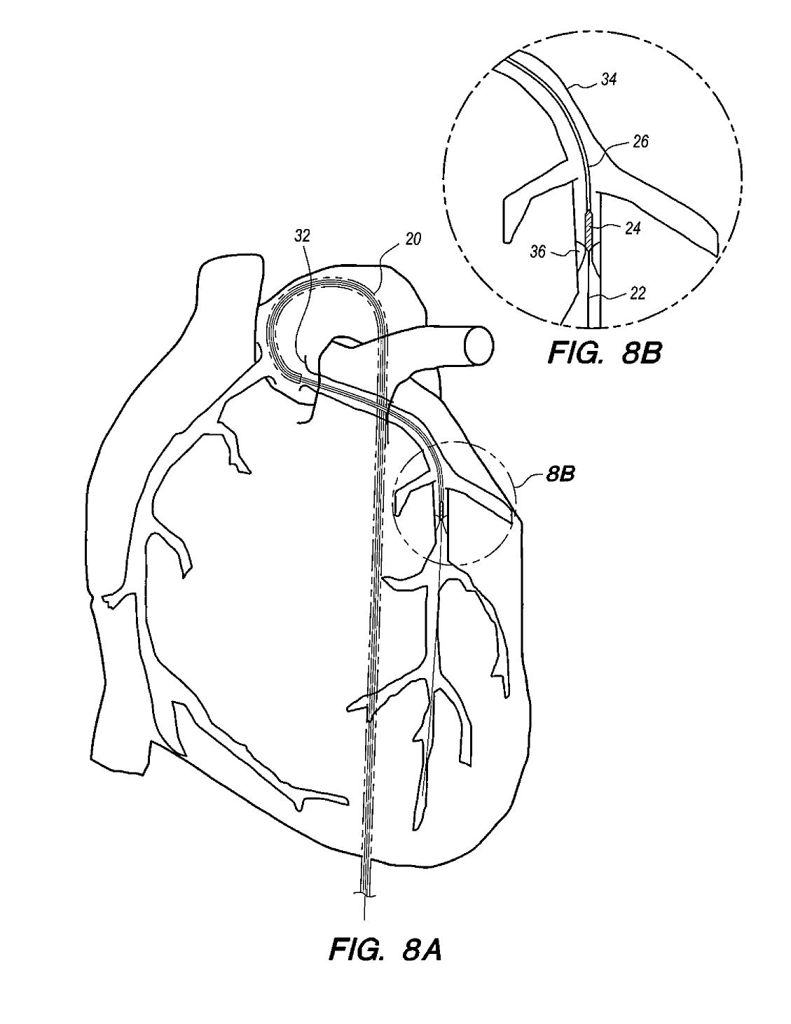

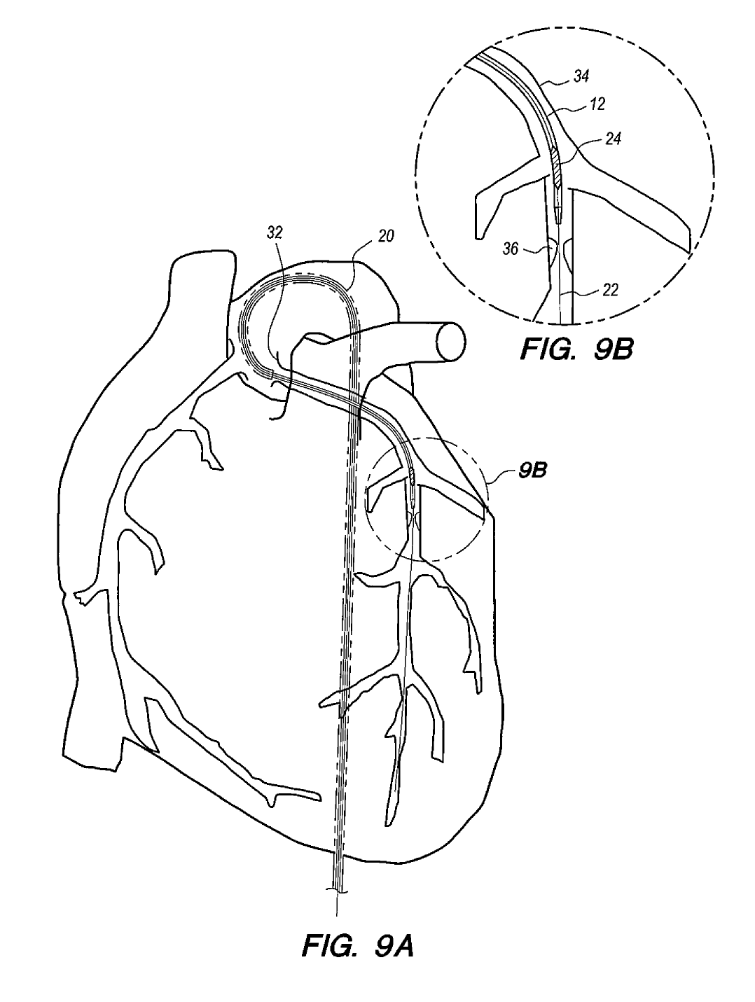

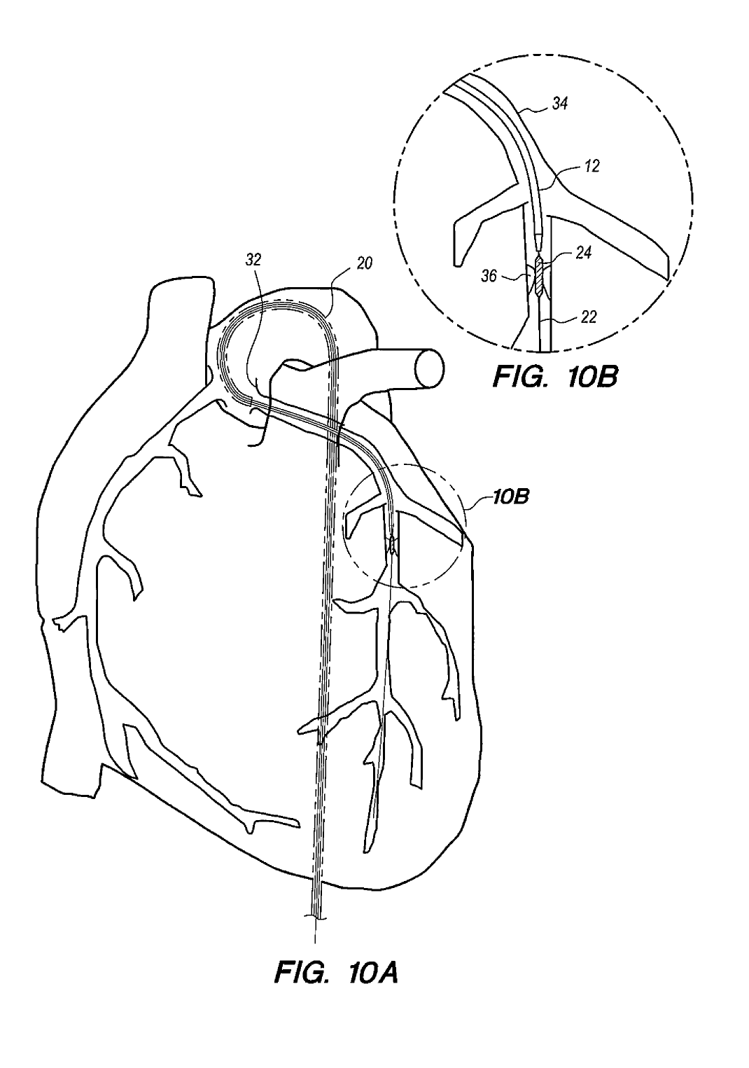

In the second use scenario depicted in FIGS. 3, 4, and 8-10, the interventional cardiology device 24 is inserted into the patient without prior insertion of the extension catheter 12. In particular, the guide catheter 20 is extended through the patient's aorta to the ostium 32 of a coronary artery 34 (shown in FIG. 3). Next, the guide wire 22 is inserted through the guide catheter lumen 30 and out of the guide catheter 20 into the coronary artery 34 to a surgical site 36, such as a lesion or stenosis (shown in FIG. 4). Then, as shown in FIG. 8, the interventional cardiology device 24 is advanced through the guide catheter lumen 30. In this scenario, the balloon catheter 26 is obstructed by the stenosis 36, such that the balloon 24 is not capable of being positioned properly. In this scenario, the extension catheter 12 is opened by the surgeon at the longitudinal slit 13 and snapped on to the proximal end of the balloon catheter 26. The extension catheter 12 is then advanced through the guide catheter lumen 30 until the distal tip 14 reaches the balloon 24. By merely abutting the balloon 24, the extension catheter 12 may provide enough support to allow the surgeon to provide sufficient force on the balloon catheter 26 in order to position the balloon 24 in the stenosis 36. Alternatively, as shown in FIG. 9, the extension catheter 12 may be advanced even further, such that the tip 14 opens and envelops the balloon 24. The extension catheter 12 continues to be advanced until the balloon 24 is disposed within the extension catheter body 15, thereby allowing the distal tip 14 to return to its original configuration. At that point, the extension catheter 12 may be maneuvered such that the distal tip 14 is at, or within, the lesion 36. The balloon catheter 26 may then be advanced out of the extension catheter 12 and positioned properly in the stenosis 36, as shown in FIG. 10.

The above description is given by way of example, and not limitation. Given the above disclosure, one skilled in the art could devise variations that are within the scope and spirit of the invention disclosed herein, including various materials to form the guide catheter extension device from and various lengths and diameters of the guide catheter extension device. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A guide catheter extension device for use with a standard guide catheter, the device comprising:
a flexible elongate extension catheter having a tapered tip portion at a distal end, an opening at a proximal end, and a body portion extending between the distal tip portion and the proximal opening, wherein the extension catheter defines a lumen extending from the tip portion to the proximal opening;
a longitudinal slit extending from the distal tip portion toward the proximal opening; and
a push rod attached to the extension catheter at the proximal opening.

2. The device of claim 1, wherein the extension catheter is formed from a resilient material.

3. The device of claim 2, wherein the resilient material is selected from the group consisting of metal, plastic, and composite structures.

4. The device of claim 3, wherein the plastic is selected from the group consisting of polyolefin, polyethylene, and polyurethane.

5. The device of claim 2, wherein the extension catheter has a first resting diameter, and is capable of deforming outward when under internal pressure by the longitudinal slit opening to define a second, larger diameter.

6. The device of claim 5, wherein the extension catheter is configured to return to the first resting diameter when the internal pressure is removed.

7. The device of claim 1, wherein the longitudinal slit extends the entire length of the extension catheter.

8. The device of claim 1, wherein the distal tip tapers from its narrowest diameter at its most distal point, and widens as it approaches the extension catheter body, and the body extends to the proximal opening at approximately the same diameter along the length of the body.

9. The device of claim 1, wherein the extension catheter tapers along its entire length from the distal tip to the proximal opening.

10. The device of claim 1, wherein the push rod is more rigid than the extension catheter.

11. The device of claim 10, wherein the push rod is formed from a material selected from the group consisting of a hypotube, stainless steel, and Nitinol tubing.

12. The device of claim 1, wherein the guide catheter extension device is longer than the guide catheter.

13. The device of claim 1, wherein the extension catheter lumen and/or the outside of the extension catheter is coated with a slippery substance.

14. The device of claim 13, wherein the slippery substance is selected from the group consisting of silicone and PTFE.

15. A method for performing an interventional cardiology procedure at a surgical site in a patient in need thereof, the method comprising the following steps:
a) inserting a standard guide catheter into a coronary artery ostium of the patient;
b) inserting a guide wire into a lumen of the guide catheter;
c) advancing the guide wire past a distal end of the guide catheter, into the coronary artery, and to the surgical site;
d) inserting a guide catheter extension device into the guide catheter lumen, such that the guide wire is disposed within a lumen of the guide catheter extension device, wherein the guide catheter extension device comprises a flexible elongate extension catheter having a tapered tip portion at a distal end, an opening at a proximal end, and a body portion extending between the distal tip portion and the proximal opening, wherein the extension catheter defines a lumen extending from the tip portion to the proximal opening, a longitudinal slit extending from the distal tip portion toward the proximal opening, and a push rod attached to the extension catheter at the proximal opening;
e) advancing the guide catheter extension device past the distal end of the guide catheter, into the coronary artery, and to the surgical site;
f) inserting an interventional cardiology device into the guide catheter extension device lumen; and
g) advancing the interventional cardiology device past the tapered tip portion and to the surgical site.

16. The method of claim 15, wherein the interventional cardiology device is a balloon catheter.

17. The method of claim 15, wherein the interventional cardiology device is a stent.

18. A method for performing an interventional cardiology procedure at a surgical site in a patient in need thereof, the method comprising the following steps:
a) inserting a standard guide catheter into a coronary artery ostium of the patient;
b) inserting a guide wire into a lumen of the guide catheter;
c) advancing the guide wire past a distal end of the guide catheter, into the coronary artery, and to the surgical site;
d) inserting an interventional cardiology device into the guide catheter lumen;
e) advancing the interventional cardiology device past the guide catheter distal end, into the coronary artery, and toward the surgical site;
f) whereupon the interventional cardiology device is blocked from reaching the surgical site, inserting a guide catheter extension device into the guide catheter lumen, such that the interventional cardiology device is disposed within a lumen of the guide catheter extension device, wherein the guide catheter extension device comprises a flexible elongate extension catheter having a tapered tip portion at a distal end, an opening at a proximal end, and a body portion extending between the distal tip portion and the proximal opening, wherein the extension catheter defines a lumen extending from the tip portion to the proximal opening, a longitudinal slit extending from the distal tip portion toward the proximal opening, and a push rod attached to the extension catheter at the proximal opening;
g) advancing the guide catheter extension device past a distal end of the interventional cardiology device and toward the surgical site; and
h) advancing the interventional cardiology device past the tapered tip portion and to the surgical site.

19. The method of claim 18, wherein the interventional cardiology device is a balloon catheter.

20. The method of claim 18, wherein the interventional cardiology device is a stent.
